(19) United States
(12) Patent Application Publication
(10) Pub. No.: US 2009/0112130 A1
(43) Pub. Date: Apr. 30, 2009

(54) Devices, systems, and methods for measurement and evaluation of tissue structures, such as breasts

(76) Inventor: Bradley P. Bengtson, Grand Rapids, MI (US)

Correspondence Address: RYAN KROMHOLZ & MANION, S.C., POST OFFICE BOX 26618, MILWAUKEE, WI, 53226, US

(21) Appl. No.: 12/287,904

(22) Filed: Oct. 15, 2008

Related U.S. Application Data
(60) Provisional application No. 60/999,331, filed on Oct. 17, 2007.

(51) Int. Cl. A61B 5/103; A61B 19/00

(52) U.S. Cl. 600/587; 128/898

(57) ABSTRACT

Devices, systems, and methods measure and evaluate breasts or other exterior tissue structures in connection with contemplated surgery, such as augmentation, revision, or reconstruction, by mechanically and/or electronically forming a three-dimensional replication of the topography of the tissue structure prior to surgery to serve as a guide for the selection of a post-operative implant.

### RELATED APPLICATIONS

This application claims the benefit of co-pending U.S. Provisional Patent Application Ser. No. 60/999,331, filed 17 Oct. 2007.

### FIELD OF THE INVENTION

This application relates generally to devices, systems, and methods for measurements and evaluation of tissue structures, such as breasts, e.g., in connection with surgical augmentation, or surgical revision, or surgical reconstruction.

### BACKGROUND OF THE INVENTION

There are a variety of measurement techniques for evaluating breasts in connection with contemplated breast surgery, such as breast augmentation, breast revision, or breast reconstruction. These techniques typically involve placing different inflatable devices or sizers inside a bra to visually aid, prior to surgery, in the selection of an implant to obtained a desired post-operative result.

Breast measurements and evaluation of breast tissues are becoming increasingly important as surgeons are switching to tissue-based implant selection methods versus just placing a breast implant of any size based on the patients or their own wishes. There are many different measurements that are obtained before and after surgery, but the most important ones that help guide a range of implant selection are the Base Width (BW) and Breast Height (BH) and Breast Projection (BP). There Are many different methods used to take these measurements, but there is a great deal of variability from surgeon to surgeon when rulers or calipers are used for taking the measurements. Breasts tend to be very asymmetric and vary greatly from side to side. Also, in reconstructive surgery, where an entire breast may be removed, matching of the contralateral side is often very challenging.

Furthermore, methods of giving patients an accurate representation of what they can expect post-operatively is difficult. This is probably the most difficult challenge to the plastic surgeon, and expectations are the key to life when it comes to cosmetic surgery results. Current methods of taking a patient's photograph and using computerized morphing has been tried, but unmet expectations may arise, along with an implied “warranty” of results that cannot always be obtained. Using a bra sizer or stuffing a bra to select implant size also does not give an accurate idea of the result, because the implant has a different shape and size when placed beneath the skin, breast and muscle, compared to placing a sizer or stuffer on top of a breast or inside a bra. An implant placed on top of the skin or breast often yields a very different result when placed beneath the tissues with no consistent correlation. There is therefore variability due to a lack of accurate measurements from surgeon to surgeon, coupled with a lack of visualization of a postoperative range of results for both the surgeon and patient to discuss.

For these and other reasons, current techniques for evaluating breasts in connection with contemplated breast surgery present problems and drawbacks.

### SUMMARY OF THE INVENTION

The invention provides devices, systems, and methods for measuring and evaluating breasts or other exterior tissue structures in connection with contemplated surgery, such as augmentation, revision, or reconstruction.

One aspect of the invention includes providing a measuring device and placing the measuring device upon a tissue structure. The measuring device may be an assembly of pins, each pin having a fixed x-coordinate position, a fixed y-coordinate position, and being movable in the z-coordinate direction as a result of placement upon a structure.

The method may include placing the measuring device upon a structure to form a three-dimensional replication of the topography of the structure.

In one embodiment the structure is an external tissue structure. In an additional embodiment the tissue structure is a breast. In an additional embodiment the structure is an implant.

The method may include using the three-dimensional replication of the topography of the breast to select a post-operative implant.

In one embodiment the three-dimensional replication of the topography of the implant provides a visual idea of the range of surgical results that are realistically possible by placement of an implant after either surgical augmentation, surgical revision, and surgical reconstruction.

The method may include providing a microprocessor coupled to pins and a display device coupled to the microprocessor.

In one embodiment the microprocessor generates image of the three-dimensional replication on a display device.

In one embodiment the measuring device includes frame, front panel, clear front panel.

In one embodiment the clear front panel includes one or more measuring scales.

In one embodiment the clear front panel includes a first measuring scale positioned along an x-axis and a second measuring scale positioned along a y-axis.

The method may include measuring the breast width. The method may include measuring the breast projection. The method may include measuring the breast circumference.

Another aspect of the invention provides a method including providing a first measuring device and providing a second measuring device, placing the first measuring device upon a first structure, wherein the placing of the first measuring device upon the first structure forms a three-dimensional replication of the topography of the first structure, and placing the second measuring device upon a second structure, wherein the placing of the second measuring device upon a second structure forms a three-dimensional replication of the topography of the second structure. The first measuring device may be an assembly of pins, each pin having a fixed x-coordinate position, a fixed y-coordinate position, and being movable in the z-coordinate direction as a result of placement upon a structure. The second measuring device may be an assembly of pins, each pin having a fixed x-coordinate position, a fixed y-coordinate position, and being movable in the z-coordinate direction as a result of placement upon a structure.

In one embodiment the structure may be a breast and the second structure may be an implant.

The method may also include comparing the three-dimensional replication of the topography of the breast to the three-dimensional replication of the topography of the implant.

### BRIEF DESCRIPTION OF THE DRAWINGS

FIGS. 1 and 2 are, respectively, a front perspective view and a rear plan view of a device for measuring and evaluating breasts or other exterior tissue structures comprising an assembly of pins, each pin having a fixed x-coordinate position, a fixed y-coordinate position, and being movable in the z-coordinate direction as a result of placement upon a tissue structure, to form a three-dimensional replication of the topography of the tissue structure, such as a breast, to serve as a guide for the selection of a post-operative implant.

FIGS. 3, 4, and 5 are, respectively, a front perspective view, a side elevation view, and a rear perspective view of the device shown in FIGS. 1 and 2, having a three-dimensional replication of the topography of a breast of the tissue structure formed as a result of contact with the breast.

FIGS. 6 and 7 are, respectively, a front perspective view and a side perspective view of the device shown in FIGS. 1 and 2, having a three-dimensional replication of the topography of an implant for a tissue structure formed as a result of contact with the implant.

FIG. 8 is a side elevation view of an alternative embodiment of a device for measuring and evaluating breasts or other exterior tissue structures comprising an assembly of telescoping pins.

FIG. 9 is a diagrammatic view of a device as shown in FIGS. 1 and 2, in association with a microprocessor to generate a virtual image of the three-dimensional replication of the topography of a tissue structure, such as a breast, to serve as a guide for the selection of a post-operative implant.

### DESCRIPTION OF REPRESENTATIVE EMBODIMENTS

Although the disclosure hereof is detailed and exact to enable those skilled in the art to practice the invention, the physical embodiments herein disclosed merely exemplify the invention that may be embodied in other specific structure. While the preferred embodiment has been described, the details may be changed without departing from the invention, which is defined by the claims.

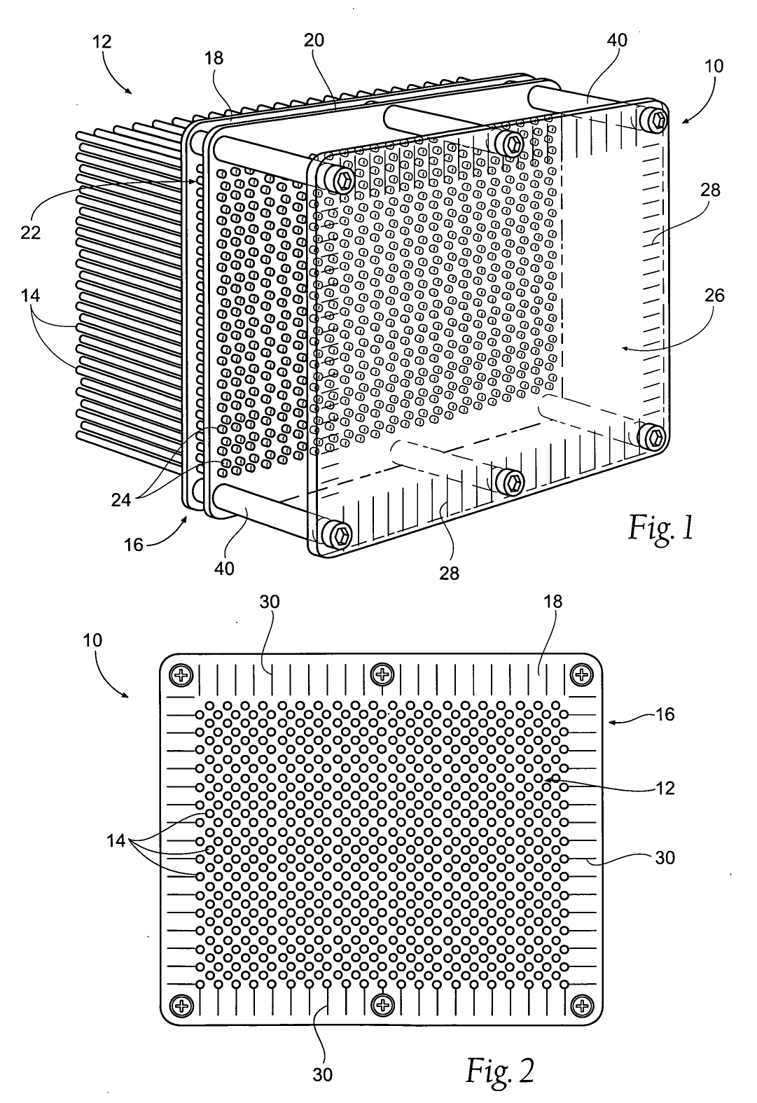

FIGS. 1 and 2 show a device 10 for measuring and evaluating breasts or other exterior tissue structures, e.g., in connection with contemplated surgery, such as augmentation, revision, or reconstruction.

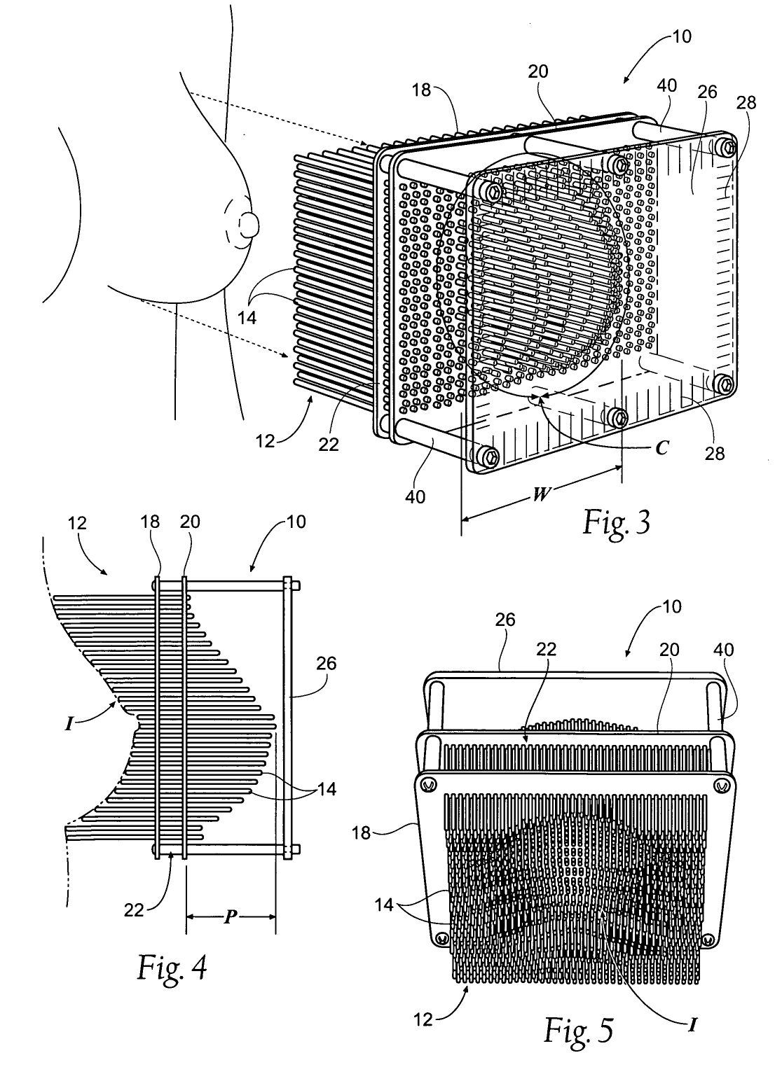

As shown in FIGS. 1 and 2, the device 10 comprising an assembly or array 12 of pins 14. The device 10 also includes a frame 16 that holds the pin array 12 in a Cartesian (X, Y, and Z) coordinate system. Each pin 14 has a fixed x-coordinate position, a fixed y-coordinate position, and is movable in the z-coordinate direction. The z-coordinate of each pin 14 is independently positionable as a result of placement upon a tissue structure, to form a desired configuration comprising a three-dimensional replication of the topography of the tissue structure, such as a breast (as FIGS. 3, 4, and 5 show).

Each pin 14 comprises an elongated stiff piece of material, such as metal or plastic. The pins 14 may comprise a variety of geometric shapes. For examples the pins may have a cylindrical shape, or other cross-sectional shapes such as squares, hexagons, etc. The diameter of the pins and the density of the pins 14 are chosen based on the intended size and detail of the tissue structure to be replicated. In general, the more pins per area, the better the resolution. However, increasing the number of pins per area inversely relates to the diameter of the pins. As the pin diameter diminishes, the strength of the pin to withstand deformation during use also diminishes.

In FIGS. 1 and 2, the frame 16, which fixes the x-coordinate and y-coordinate of the pin array 12, comprises a pair of plates 18 and 20 separated by a gap 22. Each plate 18 and 20 has a plurality of openings 24 slightly larger in diameter than the diameter of the pins 14, such that each pin 14 can move freely and independently in the z-coordinate direction, yet is nevertheless constrained within the openings 24 by the plates 18 and 20 in both the x-coordinate position and y-coordinate position. The openings 24 of the plate 18 are aligned with the openings 24 of the plate 20, such that the shaft of the pin 14 extends through the plates 18 and 20, and the pin 14 is positioned substantially perpendicular to both plates 18 and 20.

In the embodiment shown in FIGS. 1 and 2, the device 10 also includes a front panel 26 spaced by holders 40 from the frame 16. In use, the front panel 26 faces away from the tissue structure to be measured. The front panel 26 is desirably of a clear plastic material, so that the three-dimensional topography of the tissue structure replicated by the pin array 12 can be visualized (as FIG. 3 shows).

In the embodiment shown in FIGS. 1 to 4, the pins 14 have a common length, and the three-dimensional replication is formed by the different forward displacements of the pins 14 along the z-coordinate, which can be viewed through the front panel 26 as well as from the side of the device 10, as FIGS. 3 and 4 show.

The device 10 in FIGS. 1 to 4 utilizes a pin array 12 that are sized and configured to contact a patient's breast. The width of the device 10 may vary but, for typical breast measurements, can be approximately 20 cm wide. The pins 14 are spaced at about 5 mm increments, to make a three dimensional imprint I of the patient's breast (see FIG. 5), which is viewable from the side and through the front panel 26 of the device 10, as FIGS. 3 and 4 show.

The clear front panel 26 can carry indicia or markings (e.g., a company's name or advertising), and also desirably includes one or more measuring scales 28, e.g., in units of centimeters. The backmost panel 18 desirably also includes a measuring scales 30 at top and bottom, e.g., in units of centimeters. The measuring scales 28 and 30, along with the pre-established 5 mm pin width, allow a surgeon or examiner to make accurate measurements of the breast. Because the device establishes a Cartesian (X, Y, and Z) coordinate system, the measurements will be much more standardized between examiners than current methods, using, e.g., calipers and/or rulers.

Using the device 10, measurements from the breast imprint may include breast base width W (the most critical breast measurement) (see FIG. 3), breast projection P (see FIG. 4), and breast circumference C (see FIG. 3), both before and after surgery.

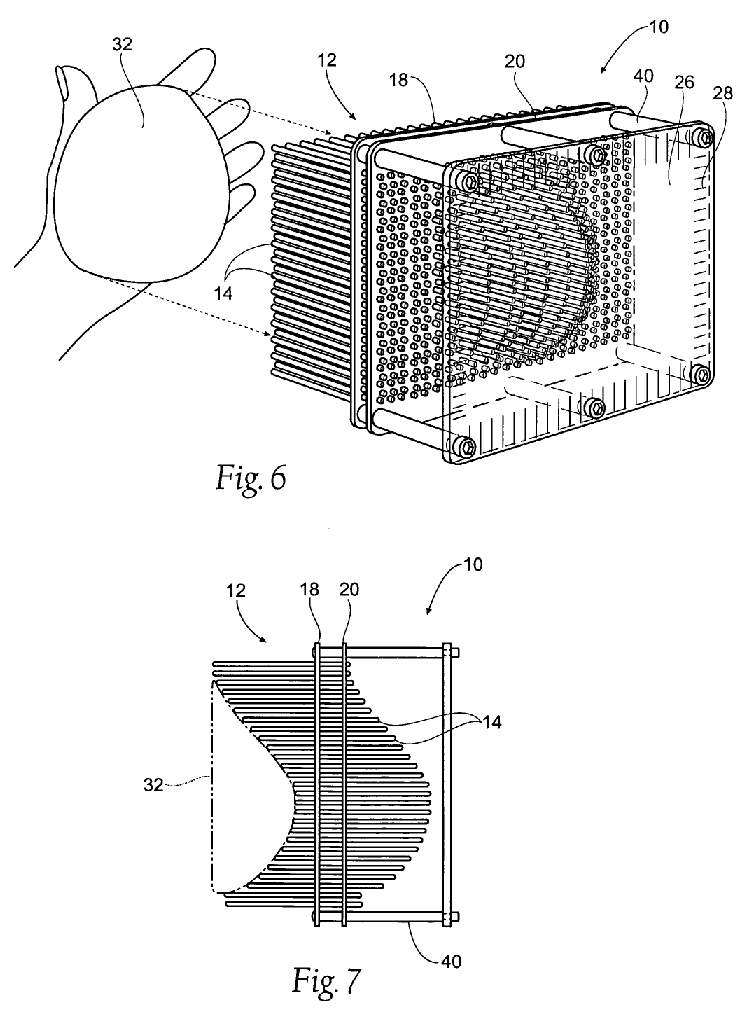

After the breast has been placed in the device 10, an imprint obtained, and measurements taken, the thickness of the device 10 accommodates the placement of various size implants 32, also expanding the pins and giving the patient and surgeon a better 3-dimensional view of how the final breast may look (see FIGS. 6 and 7). This, in conjunction with before and after photographs of other patients with similar breast measurements, can give an accurate visual idea of the range of surgical results that are realistically possible.

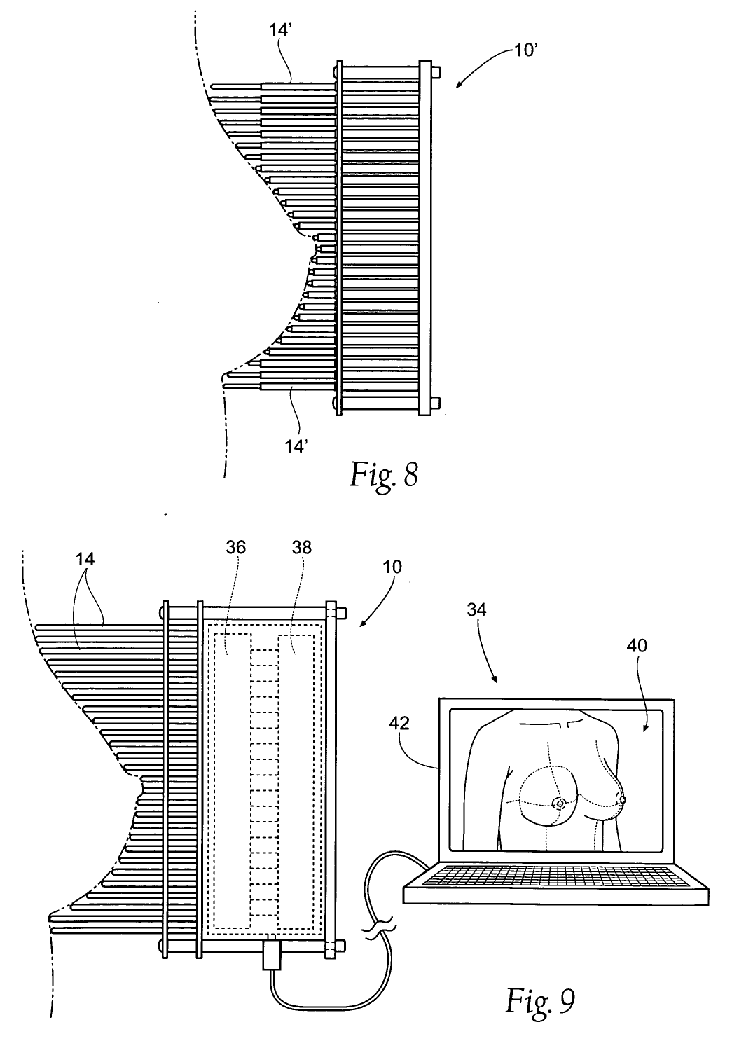

As shown in FIG. 8, the device 10′ may include an array of pins 14′ that collapse or telescope in on themselves, as shown in FIG. 5. In this arrangement, after the breast is placed in the device 10′, an imprint obtained, and measurements taken, various size implants may also be placed to give the patient and surgeon a better three-dimensional view of how the final breast may look.

As shown in FIG. 9, the device 10 can form a part of a microprocessor augmented tissue measurement system 34. The system includes an array of proximity sensors 36 associated with the pins 14. The proximity sensors 36 can comprise, e.g., infrared proximity sensors, or capacitive proximity sensors, or inductive proximity sensors, that generate signals as a function of the distance between the pins 14 and the sensors 36. In this arrangement, displacement of the pins 14 generates a pattern of output signals from the sensors 36 indicative of the physical configuration of the imprint. The pattern of output signals from the sensors 36 can be processed according to pre-programmed algorithms residing on a microprocessor 38, to output the imprint parameters expressed in terms of the desired measurements, e.g., breast base width, breast projection, and breast circumference. The pattern of output signals from the sensors 36 can also be processed by digital display algorithms residing on the microprocessor 38 to generate a virtual graphic image 40 on a display device 42. The virtual graphic image 40 conforms to the physical parameters of the breast imprint taken by the device 10, which desirably can then be viewed, manipulated, and rotated 3-dimensionally. The system 34 can also generate virtual images of implants of varying sizes behind or superimposed on the virtual image of the breast imprint, to actually show on the display device 42 the augmented size of the implant, which also may be rotated to better visualize the breast and implant 3-dimensionally.

The foregoing is considered as illustrative only of the principles and technical features of the invention. Furthermore, since numerous modifications and changes will readily occur to those skilled in the art, it is not desired to limit the invention to the exact construction and operation shown and described. While a representative embodiment has been described, the details may be changed without departing from the principles and technical features of the invention.

We claim:

1. A method comprising:
providing a measuring device, the measuring device comprising an assembly of pins, each pin having a fixed x-coordinate position, a fixed y-coordinate position, and being movable in the z-coordinate direction as a result of placement upon a structure; and
placing the measuring device upon a tissue structure.

2. The method of claim 1 wherein the placing of the measuring device upon a structure forms a three-dimensional replication of the topography of the structure.

3. The method of claim 2 wherein the structure is an external tissue structure.

4. The method of claim 3 wherein the tissue structure is a breast.

5. The method of claim 4 further comprising using the three-dimensional replication of the topography of the breast to select a post-operative implant.

6. The method of claim 2 wherein the structure is an implant.

7. The method of claim 6 wherein the three-dimensional replication of the topography of the implant provides a visual idea of the range of surgical results that are realistically possible by placement of an implant after one of surgical augmentation, surgical revision, and surgical reconstruction.

8. The method of claim 2 further comprising providing a microprocessor coupled to pins and a display device coupled to the microprocessor.

9. The method of claim 8 wherein said microprocessor generates image of the three-dimensional replication on a display device.

10. The method of claim 2 wherein the measuring device includes frame, front panel, clear front panel.

11. The method of claim 10 wherein the clear front panel includes one or more measuring scales.

12. The method of claim 11 wherein the clear front panel includes a first measuring scale positioned along an x-axis and a second measuring scale positioned along a y-axis.

13. The method of claim 12 wherein the structure is a breast.

14. The method of claim 12 further comprising measuring the breast width.

15. The method of claim 12 further comprising measuring the breast projection.

16. The method of claim 12 further comprising measuring the breast circumference.

17. A method comprising:
providing a first measuring device, the measuring device comprising an assembly of pins, each pin having a fixed x-coordinate position, a fixed y-coordinate position, and being movable in the z-coordinate direction as a result of placement upon a structure;
providing a second measuring device, the measuring device comprising an assembly of pins, each pin having a fixed x-coordinate position, a fixed y-coordinate position, and being movable in the z-coordinate direction as a result of placement upon a structure;
placing the first measuring device upon a first structure, wherein the placing of the first measuring device upon the first structure forms a three-dimensional replication of the topography of the first structure; and
placing the second measuring device upon a second structure, wherein the placing of the second measuring device upon a second structure forms a three-dimensional replication of the topography of the second structure.

18. The method of claim 17 wherein the first structure is a breast and the second structure is an implant.

19. The method of claim 18 further comprising comparing the three-dimensional replication of the topography of the breast to the three-dimensional replication of the topography of the implant.
